(19) United States
(12) Patent Application Publication
(10) Pub. No.: US 2005/0203428 A1
(43) Pub. Date: Sep. 15, 2005

(54) Device and method for determining coronary blood flow

(75) Inventor: William V. Judy, Bradenton, FL (US)

Correspondence Address: BROWDY AND NEIMARK, P.L.L.C.;624 NINTH STREET, NW, SUITE 300, WASHINGTON, DC, 20001-5303, US

(73) Assignee: IMPEDANCE VASCULAR IMAGING PRODUCTS, LLC, Bradenton, FL (US)

(21) Appl. No.: 10/800,018

(22) Filed: Mar. 15, 2004

(51) Int. Cl. A61M 1/00; A61B 5/02

(52) U.S. Cl. 600/506

(57) ABSTRACT

The present invention relates to a device and method for measuring a blood flow through a coronary system of a heart. It was realized that said blood flow causes a temporary change in the impedance of the coronary system, in the form of a peak in a first time-derivative of the impedance signal. The method comprises measuring an impedance signal across the body region containing the coronary system as a function of time, determining a first derivative of the impedance signal with respect to time, and calculating the blood flow from a peak height of a certain peak signal in said derivative impedance signal. The device comprises a bioimpedance measuring device adapted for the method.

### FIELD OF THE INVENTION

The present method generally relates to a device method for determining on a beat-to-beat basis at least the blood flow through at least a selected half of the coronary artery system of a beating heart of a mammal, in particular a human being, during a beat of the heart, comprising or using a bioimpedance measuring device.

### BACKGROUND OF THE INVENTION

Cardiac monitoring is the measuring of various quantities related to the functioning of the human or animal heart. An important quantity is the pulsatile (i.e. non-laminar beatwise), blood flow through the left and/or right coronary artery. These coronary arteries supply oxygen, or generally blood, to the heart muscle. When, for any reason, this blood flow is diminished or impeded (stenosis), serious heart failure may be observed. Even when stenosis exists unnoticed, e.g. when no physical exercise is performed, the risk of a coronary artery being blocked by a thrombus is highly increased. These and other healthcare risks are the source of extensive and expensive healthcare.

In the art, various methods are used to determine the blood flow in a artery. In some methods, a catheter is introduced into a blood vessel. This is however very risky and bothersome for the patient. Furthermore, supervision by a physician is necessary.

Furthermore, U.S. Pat. No. 3,835,840 describes an apparatus and method for noninvasively measuring blood flow in an artery. In this method, a bioimpedance measuring device is applied to a body section such as a limb the blood flow through a main artery of which is to be determined. However, this method or device cannot be used for measuring the blood flow through a coronary artery because no method is indicated how to determine the signal that corresponds to the flow through a coronary artery. The reason is that when a bioimpedance measuring device is applied to the chest, various signals play a role in establishing the bioimpedance signal, viz. blood flow through the aorta, venae cavae, atria, etc.

Thefore there exists a need for a device and a method for determining on a beat-to-beat basis the blood flow through at least a selected half of the coronary artery system of a beating heart, that are furthermore noninvasive, of low risk to the patient and without the need of a physician's supervision.

### SUMMARY OF THE INVENTION

One aspect of the present invention is a device for determining at least the blood flow through at least a selected half of a coronary artery system of a beating heart of a mammal, in particular a human being, during a beat of the heart, comprising a bioimpedance measuring device for measuring an impedance signal Z that depends on the blood flow through at least the selected half of the coronary artery system, which bioimpedance measuring device comprises a current source, supply electrodes, one or more upper measuring electrodes, one or more lower measuring electrodes and measuring means, and further comprising processing means which are connected to the measuring means, for processing of at least a value of the impedance signal Z, and for determining a first time-derivative dZ/dt of the impedance signal Z, wherein
the processing means further comprise means for separating from the first time-derivative dZ/dt a peak signal PS corresponding to the selected half of the coronary artery system, which peak signal PS lies within a time interval between the beginning of diastole of the heartbeat and the end of a second peak signal PS2 of the first time-derivative dZ/dt which occurs second after the beginning of diastole, and wherein the processing means are designed to determine the blood flow from the peak signal PS. The peak signal PS is either PS1, i.e. the first of the two peak signals, if the selected half is the left coronary artery system, or it is PS2, i.e. the said second peak signal, if the selected half is the right coronary artery system. This will be elucidated below.

The current source, supply electrodes, and measuring electrodes may be standard parts of an ordinary bioimpedance measuring device. This will be further elucidated with respect to the description of the preferred embodiment.

The processing means may be a programmed computer, an analog or digital electronic circuit etc. The processing means are designed such that they can determine a first time-derivative of an input signal. The procedure and devices therefor are well-known in the art and will not be elucidated further.

The separation from the first time-derivative dZ/dt of a peak signal PS is to be understood as selecting that part of the first time-derivative signal that corresponds to the blood flow through the coronary artery or arteries. It has been found by the inventor that the first two peaks during diastole in the first time-derivative signal correspond to the left, the right coronary artery blood flow, respectively. Diastole may be determined by any known means or method, but this will be further explained later on.

Determining the blood floww here means that information about the blood flow is obtained. E.g. left and right coronary flow may be compared with each other. Left flow should be about three times as large as right flow, in healthy patients. It is also possible to compare measured signals over time, to see if some blocking has occurred or has been removed, etcetera. More quantitative information may either be obtained by carrying out a comparative quantitative measurement with some other method or means, i.e. calibrating, or with one of the preferred embodiments, to be described hereinbelow.

In a preferred embodiment of the device according to the invention, the processing means are designed to determine the blood flow through the selected half of the coronary artery system from a first peak signal PS1 which occurs first after the beginning of diastole of the heart beat, in the case that the selected half of the coronary artery system is the left half of the coronary artery system of the heart. The first peak signal PS1 corresponds to the blood flow through the left half of the coronary artery system. It comes first because of a lower resistivity to blood flow.

In another preferred embodiment the processing means are designed to determine the blood flow through the selected half of the coronary artery system from the second peak signal PS2 which occurs second after the beginning of diastole of the heart, in the case that the selected half of the coronary artery system is the right half of the coronary artery system of the heart. The second peak signal PS2 correspond to the blood flow through the right half of the coronary artery system. Due to higher resistance this peak signal PS2 occurs 10-20 ms after PS1.

Both signals occur during early diastole, i.e. in the first part of the phase of the heartbeat in which the heart muscle relaxes. In fact, diastole begins with closing of the aortic valve. The beginning of diastole may be determined in any way known in the art. Preferably, it is determined from the dZ/dt signal, in that diastole begins at the moment where dZ/dt assumes a minimum value following a maximum value. This will be further elucidated with reference to the drawings. It is to be noted that in practice, out of custom, the Z signal, and hence the first time-derivative dZ/dt signal, is inverted. Hence, when the pure Z signal is measured, in fact any minimum value as defined or mentioned in this document corresponds to a maximum value in the physical reality. In other words, whenever the term mimimum value is used in this document, this corresponds to a minimum value on a screen or in a display, which has been inverted with respect to the physical reality.

Advantageously, the processing means are further designed to determine the duration TCAFT that corresponds to the total coronary artery flow time of the coronary artery system, and further to determine the blood flow volume CQ through the selected half of the coronary artery system during the heart beat, according to
CQ=C/(Z₀H)²·TCAFT·(MCdZ/dt), in which
C=a predetermined constant expressed in Ohm·cm³,
MCdZ/dt=maximum value of the separated peak signal PS in Ohm/s, and
Z₀H=value of the impedance in Ohm of the thorax of the mammal at the time of MCdZ/dt, during the heartbeat,
wherein TCAFT is determined as the time between the beginning of the first peak signal PS1 and the end of diastole of the heartbeat, and
wherein the measuring electrodes have been placed at the midneck region and at the xiphoid junction of the mammal. Hence CQ is expressed in cm³ or milliliters. The end of diastole may e.g. be determined as the time within a heartbeat, at which the dZ/dt signal assumes minimum value just preceding the maximum value.

Hence the blood flow volume through the left half of the coronary artery system is determined as LCQ is C/(Z₀H)²·TCAFT·(MC_{L} dZ/dt), and accordingly for the right half the letter L is to be replaced by letter R.

The units given here are the usual units. It is of course possible to take different units. Then a conversion factor should be introduced to account for the difference in units. This is a standard procedure, which may be readily applied by the person skilled in the art.

In a preferred embodiment, the constant C=ρ·(Lm)², in which ρ=the specific resistivity of the blood of the mammal expressed in Ohm·cm, and Lm=the myocardial distance from an aortic valve of the heart to the apex of the heart expressed in cm. Although basically the constant C need not be determined from the resistivity of the blood, it has been found that this choice of the constant give a very accurate result. Furthermore, the taking of a sample of the blood of a patient is standard hospital routine, and is no great burden for the patient.

Preferably, the resistivity ρ is set equal to ρ=53.2·e^{0.022·Hct}, wherein Hct is the dimensionless hematocrit value of a sample of the blood of the mammal. The hematocrit value of blood is the ratio of the volume of blood cells in a sample and the total volume of the sample. In normal men, this ratio is about 0.45, and in normal women about 0.42. Again, it is not necessary to determine the hematocrit value, but this is a routine measurement. Nevertheless, the resistivity ρ may be determined according to any method known in the art.

In an expedient embodiment of the device according to the present invention, the processing means are further designed to determine a heart rate in beats per unit time, and further designed to determine a blood flow volume CAQ per unit time through the selected half of the coronary artery system, according to CAQ=CQ·HR. The means for determining the heart rate may be any means known in the art. Examples are a counter system that counts on the basis of an electrocardiogram, or a heart sound detection system. Preferably however, the heart rate in determined by means of a system that counts on the basis of the measured impedance signal Z. The heart rate HR may easily be determined from the time between consecutive maximum values of the first time-derivative dZ/dt. The heart rate is usually expressed in beats/minute, although some other unit may be used as well, provided the correct conversion factor is introduced in the formula.

Advantageously, the processing means are further designed to determine the total blood flow volume through the coronary artery system as the sum of the coronary blood flow volume for the left half of the coronary artery system and the coronary blood flow volume for the right half of the coronary artery system. Obviously, if both the left and the right coronary blood flows are known, the total coronary blood flow may be determined.

Another aspect of the present invention is a method for determining at least the blood flow through at least a selected half of a coronary artery system of a beating heart of a mammal, in particular a human being, during a beat of the heart, comprising the steps of
applying upper and lower measuring electrodes to the body of the mammal, wherein the selected half of the coronary artery system is completely between the upper and lower measuring electrodes,
measuring an impedance signal Z which depends on the blood flow through the selected half of the coronary artery system, by means of a bioimpedance measuring device which is connected to the upper and lower measuring electrodes,
determining a first time-derivative dZ/dt of the impedance signal Z,
separating from the first time-derivative dZ/dt a peak signal PS which corresponds to the selected half of the coronary artery system, which peak signal PS lies within a time interval between the beginning of diastole of the heart during the heartbeat and the end of a second peak signal PS2 of the first time-derivative dZ/dt which occurs second after the beginning of diastole, and
determining the blood flow from the separated peak signal PS.

Although the upper and lower measuring electrodes may be applied to any part of the body provided the rule stated here is obeyed, most of the time a certain standard is followed. In this standard, the upper measuring electrode or electrodes are applied at the midneck region, whereas the lower measuring electrode or electrodes are applied at the xiphoid junction. Of course the supply electrodes have also been applied to the body in such a position that the measuring electrodes are between the upper and lower supply electrodes. Ordinarily, the upper supply electrode or electrodes are applied at the forehead level, and the lower supply electrode or electrodes are applied at the abdominal level. These particular locations are used for easy referencing and to be able to compare results with previously measure result. However, in particular cases these locations may be chosen differently, e.g. when a wound of the patient necessitate this. However, obviously all of the heart must be comprised between the upper and lower measuring electrodes.

Preferably, the blood flow through the selected half of the coronary artery system is determined from a first peak signal PS1 which occurs first after the beginning of diastole of the heart beat, in the case that the selected half of the coronary artery system is the left coronary artery system of the heart.

As stated above, the first peak signal PS1 correspond to the blood flow through the left half of the coronary artery.

In another preferred embodiment of the method according to the invention, the blood flow through the selected half of the coronary artery system is determined from the second peak signal PS2 which occurs second after the beginning of diastole of the heartbeat, in the case that the selected half of the coronary artery system is the right coronary artery system of the heart. The second peak signal PS2 corresponds to the blood flow through the right half of the coronary artery system, and occurs some 10-20 ms after PS1.

In an expedient embodiment, a blood flow value CQ during the heartbeat through the selected half of the coronary artery system during the heart beat is determined, according to
CQ=C/(Z₀H)²·TCAFT·(MCdZ/dt), in which
C=a predetermined constant,
MCdZ/dt=maximum value of the separated peak signal PS,
Z₀H=value of the impedance of the thorax of the mammal at the time of MCdZ/dt, during the heartbeat, and
TCAFT=total coronary artery flow time=time between the beginning of the first peak signal PS1, and the end of diastole of the heartbeat,
wherein the upper measuring electrodes, lower measuring electrodes, respectively, have been applied at the middle neck region, at the xiphoid junction of the sternum of the mammal, respectively. Units are the same as with the device.

Here, the upper and lower measuring electrodes have been applied at the locations described above. It is possible to use different locations, however, then it must be borne in mind that the predetermined constant may be adjusted.

Preferably, the constant C is set equal to C=ρ·Lm², in which
ρ=specific resistivity of the blood of the mammal, and
Lm=the myocardial distance from an aortic valve of the heart to the apex of the heart.

Advantageously, the resistivity ρ is set equal to ρ=53.2·e^{0.022·Hct}, wherein Hct is the hematocrit value of a sample of the blood of the mammal.

Advantageously, the method according to the invention further comprises the steps of determining the heart rate HR in beats per unit time, and determining the blood flow volume CAQ per unit time throught the selected half of the coroanry artery system, according to CAQ=CQ·HR. Again, the heart rate may be determined according to any known way, and this method holds for both the left half and the right of the coronary artery system.

Expediently the method further comprises the step of determining the total value of the blood flow volume through the coronary artery system during the heartbeat as the sum of the blood flow volume through the left half of the coronary artery system and the blood flow volume through the right half of the coronary artery system.

In another expedient embodiment of the method according to the invention, the method further comprises the step of determining the total value of the blood flow volume per unit time through the coronary artery system during the heartbeat as the sum of the blood flow volume per unit time through the left half of the coronary artery system and the blood flow volume per unit time through the right half of the coronary artery system. The chosen time-unit for the total blood flow through the coronary arteries depends on the time-unit that was chosen to determine the blood flow through either half of the coronary artery system, i.e. to express the heart rate. If for example the heart rate was expressed in beats per minute, then the blood flow is expressed in vol/min, e.g. ml/min. This may however easily be converted into ml/s or l/h etc.

### BRIEF DESCRIPTION OF DRAWINGS

FIG. 1 shows a diagrammatic view of the device according to the invention, in use on a diagrammatic patient.

FIG. 2 shows diagrammatically unexemplary measured signal, representing _{Δ}Z, which is the difference in the impedance with respect to the basic value.

FIG. 3 shows the first time-derivative of the _{Δ}Z signal.

FIG. 4 shows a phonocardiogram-signal.

FIG. 5 shows an electrocardiogram-signal.

FIG. 6 shows an enlarged detail of FIG. 3.

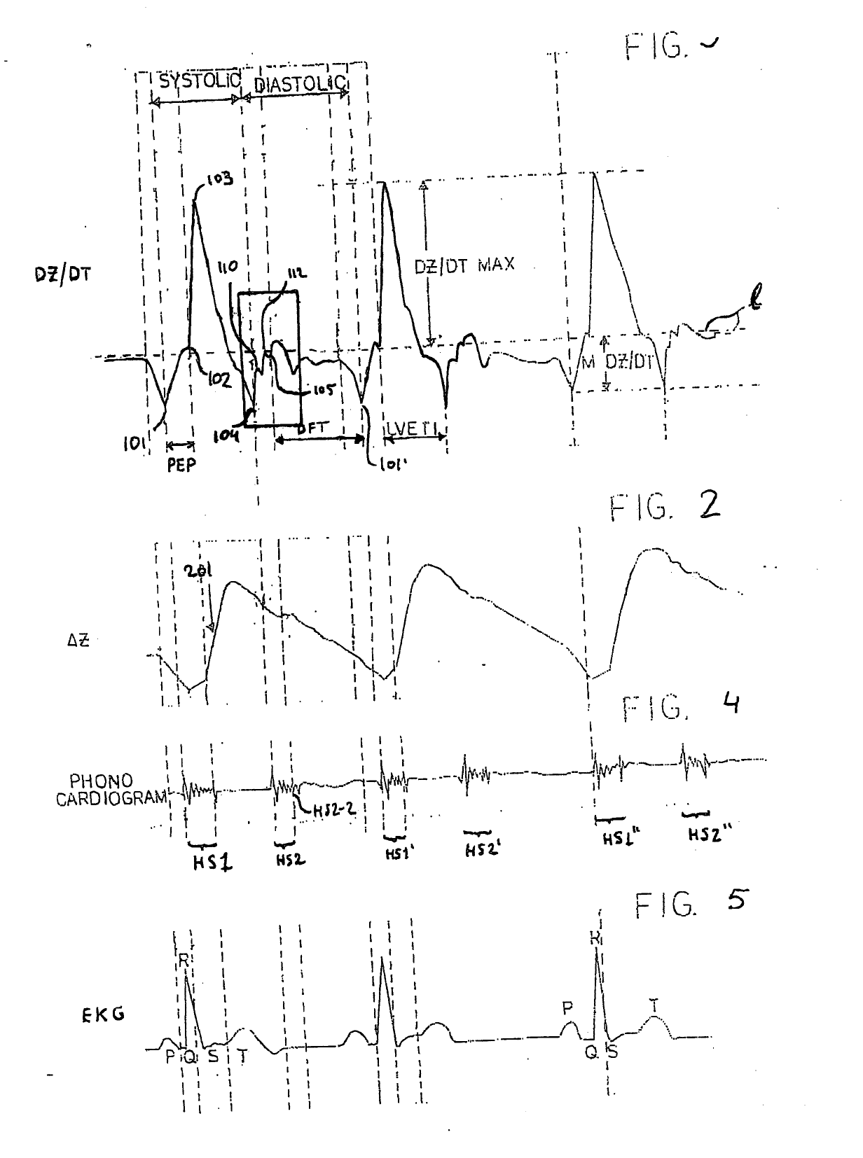

FIG. 2 through 5 are taken and represented of the same time-axis, i.e. in all four figures the same point in time is represented by the same position of the horizontal axis. This is further indicated by the vertical dashed line.

### DETAILED DESCRIPTION OF THE PREFERRED EMBODIMENT

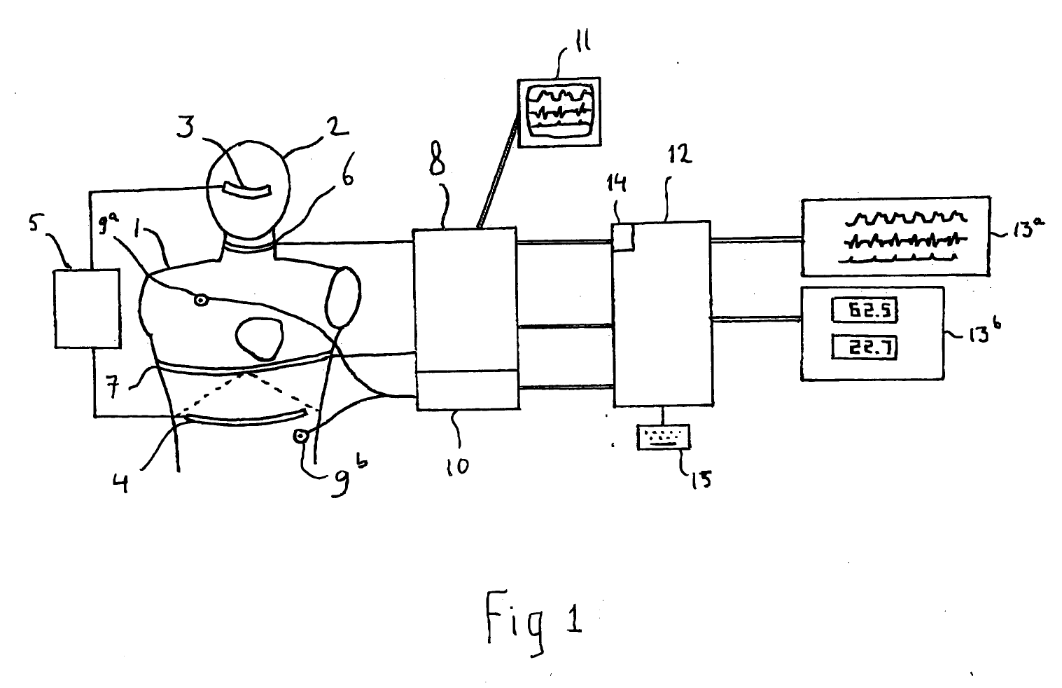

In FIG. 1 there is shown diagrammatically the upper part 1 of a body of a human being, including a head 2. One or more upper supply electrodes 3 are applied to the forehead, and one or more lower supply electrodes 4 are supplied to the upper part 1 of the body at waist level. A current source 5 is connected to both the upper supply electrodes 3 and the lower supply electrodes 4.

One or more upper measuring electrodes 6 are applied to the body at the mid neck region, and one or more lower measuring electrodes 7 are applied at the height of the xiphoid junction of the sternum. The upper 6 and lower 7 measuring electrodes are connected to a bioimpedance measuring device 8. Furthermore, first 9a and second 9b EKG-electrodes are applied to the body, and are connected to an EKG-measuring means 10. Furthermore, phonocardiogram measuring means could be applied to the body, at the 3 intercostal space, just left of the sternum. For clarity in the figure, these phonocardiogram measuring means are not shown.

Both the bioimpedance measuring means 8 and the EKG-measuring means 10 are connected to a display 11 and to a processing unit 12. In its turn, the processing unit 12 is connected to a monitor 13a, 13b.

The processing unit 12 is optionally provided with invertor means 14 and/or a keyboard 15.

The one or more upper supply electrodes 3 and/or the one or more lower supply electrodes 4 are e.g. metallic strip electrodes or spot electrodes. Strip electrodes may be elongate strips passing at least half way, and preferably all the way around the body of the patient, for example in the form of circumferential electrodes. Spot electrodes may be of a substantially round and square shaped or the like. In the case of strip electrodes it is preferred to take only one electrode for each of the supply and/or measuring electrodes 3, 4, 6, 7. In the case of spot electrodes, it is possible to use either one, but preferably two or more electrodes, for each of the supply and/or measuring electrodes.

They may be coated with aluminum or some other electrically conducting material. They may be applied to the body with the help of an electrically conducting gel. This not only helps to reduce the transitional impedance between electrodes and skin, but also allows that the patient moves, to some extent, the body part to which the electrodes have been applied, without this having an adverse effect on the measurements.

The supply electrodes 3, 4 serve to establish a current field through at least the thorax of the patient. To that end, they should be applied such that at least the thorax, with the heart, is comprised between the upper supply electrodes 3 and the lower supply electrodes 4. This means that the upper supply electrodes should be applied at least as high as the neck level, and the lower supply electrodes at least as low as the level of the xiphoid junction. Preferably however, the upper supply electrodes 3 are supplied at the level of the forehead, because then there will be little or no edge effects and a more homogenous current field. Moreover, there will very likely be no interference with other electrodes or medical apparatus applied to the body of the patient. Preferably, the lower supply electrodes are applied at abdominal level, for equivalent reasons. It should be noted that other locations of application are allowable. However, it may then be necessary to first do a calibration, to correct for possible changes in numerical constants. Such changes may be due to for example geometrical effects, such as a different current-field through the thorax, caused by a different electrode configuration. As with all measuring instruments, such calibration is advisable even when the configuration has not been changed.

The current source 5 is, although not strictly necessary, preferably a constant current source. This means that the set current remains constant, independent of changes in the impedance through which the current is sent. This greatly improves the accuracy and user-friendliness over non-constant-current sources. Non-constant-current sources may be used if every measurement of the impedance signal Z is corrected for, i.e. divided by, the actual value of the current at the time of the measurement. This is however bothersome, and may decrease accuracy.

The constant current source delivers a current of physiologically safe properties. This means that the used frequency lies in a range within which there is little or no interference with electrical body processes. Advantageously, this frequency range is from about 60 kHz to about 200 kHz, and preferably from about 70 kHz to about 100 kHz. It also means that the current is less than about 5 mA, rms value, and preferably between about 2 and about 4 mA.

The measuring electrodes 6, 7 can be of a type equivalent to the supply electrodes 3, 4, i.e. of the strip type, of the spot type or of mixed type.

The current through the body generates a voltage difference across the body that depends on the magnitude of the current and the impedance of the parts of the body (blood, tissue, etc.) between the electrodes. In fact, Ohm's law is applicable, and the relationship impedance=voltage divided by current may be used to determine the impedance.

By applying the measuring electrodes 6, 7 at the height of the middle neck region and the xiphoid junction of the sternum, respectively, the voltage difference across the thorax can be measured with the help of the measuring means 8. With the help of this voltage signal, the impedance of the thorax, and variations thereof, can be determined. For this reason, and because the voltage signal that is picked up by the measuring electrodes depends on the magnitude of the applied current, the signal is hereinbelow referred to as the impedance signal.

Even though the preferred locations of application are as described above, the measuring electrodes 6, 7 may be applied to the body at a different height, but under the following restrictions. Firstly, the thorax with the heart must be comprised between the measuring electrodes 6, 7. Secondly, the measuring electrodes 6, 7 must be applied between the upper supply electrodes 3 and the lower supply electrodes 4. Thirdly, a calibration may be necessary to account for possible changes in the geometry of the measuring set-up. E.g. if the measuring electrodes are applied to the body somewhat higher or lower, then more or less thorax tissue, blood etc. contributes to the impedance, while the contribution of the coronary arteries remains the same. This may lead to some changes in the numerical constants that are used in the method according to the invention.

Advantageously, there should be a distance of at least 2 cm between any supply electrode 3, 4 and any measuring electrode 6, 7 to prevent interference effects and to suppress edge effects. This restriction is more severe in the case of supply electrodes of the spot type than in the case of strip-type supply electrodes. Preferably, this distance is larger, because in persons with short necks, babies, etc. it becomes impossible to place the upper electrodes correctly. Advantageously, the upper supply electrode(s) is/are placed on the forehead. Correspondingly, the lower measuring electrode(s) is/are placed at the abdominal level.

The bioimpedance measuring means 8 may be any standard bioimpedance measuring device. In practice, this will be a volt-meter or oscilloscope, but any other means for measuring a voltage difference would suffice as well. The bioimpedance measuring means determine the value of the voltage between the upper measuring electrodes 6 and the lower measuring electrodes 7. For the purpose of this invention this voltage signal is referred to as the impedance signal Z, whether or not it has been converted to the underline impedance value. If not, the voltage signal should be divided by the current in a later calculation.

The bioimpedance measuring means 8 may be connected to an optional display 11. This display 11 can show the measured impedance signal as a function of time. With the help of the display 11 the person operating the device according to the invention can see if the measured impedance signal can be relied upon, i.e. it does not show artefacts. For example, it would be possible for there to be a loose connection or noise. This could result in a measurable but useless signal or a signal with spikes or other physiologically meaningless features. This is not always visible when only a read-out of the value of the impedance signal is considered, but much more easily recognized when looking at the display 11. It should be noted that whenever in this document the term “output”, “outputting” etc. is used, this may mean the displaying on a screen, display, etc., but also the outputting of a signal or value to some further device, e.g. for storage or further processing.

Reference numerals 9a and 9b indicate two optional electrocardiogram (EKG)-electrodes, which are connected to EKG-measuring means 10. In FIG. 1 the EKG-electrodes 9a and 9b are applied to the body in a so-called lead II configuration. They serve to measure the electrical activity of the heart, and can be of any normally used type. The EKG measuring means 10 can be connected to the display 11, in order to be able to visually check the electrical activity of the heart. The EKG-signals as thus determined may be used for several purposes. For example, they can be used for timing purposes, that is, to assign certain signals or points in time to a certain part of the cardiac cycle. Furthermore, it is possible and preferred to establish the heart rate from the EKG-signal. It must be stressed however, that the EKG is not a necessary part of the device according to the invention. The heart rate, e.g., may also be determined from the impedance signal, or by acoustic means.

The impedance measuring means 8, and optionally the EKG-measuring means as well, can be connected to the processing unit 12. Basically, the processing unit 12 is a computer with a computer programme. The processing unit may however come in the form of an electronic circuit or the like, that has been programmed to carry out the method according to the invention. The processing unit 12 may comprise convertor means to sample and digitize the measured impedance signal and turn the analog signal into a digital signal which can be processed by the computer. An analog way of processing the signals is however possible too, though it is very much less flexible, e.g. as to entering of patient related data.

The processing unit 12 further comprises a differentiator (not shown). The differentiator determines the first derivative with respect to time of the measured impedance signal Z, which first time-derivative signal is referred to as dZ/dt. This can either be performed on the analog impedance signal in an analog way by a suitable circuit, or on the digitized impedance signal in a digital way by a suitable programme. It is noted here that for the purpose of subsequent calculations, it makes no difference whether ΔZ or Z are used. Hence they may be interchanged in the text. However, the quantity Z₀ refers to a definite quantity, and here there should be and can be no interchanging with some differential value ΔZ₀.

In the preferred embodiment, the device as a whole comprises, as is customary, an invertor 14 which inverts the signal dZ/dt. It is expressly stated here that wherever a maximum or a minimum value of the signal dZ/dt is mentioned, this is meant to be a maximum or minimum value of the inverted signal dZ/dt. In reality this corresponds with a minimum value, a maximum value of the first time-derivative, respectively. If no invertor 14 is used, that is, if the non-inverted signal dZ/dt is used to do the calculations according to the method of the invention, it should be borne in mind that the words “maximum” and “minimum” in the claims should be interchanged. It may be possible to show and/or measure the impedance signal as a difference _{Δ}Z from an average value Z_{avg}. In this case it is possible to invert only _{Δ}Z, after which the signal dZ/dt is already inverted with respect to the real physical value thereof.

The measured signals, _{Δ}Z, dZ/dt, EKG, etc., whether or not inverted, may be displayed on a monitor 13a. Not only may the monitor 13a serve as a visual check of the quality of the signals, as stated above in connection with the other signals, but it is also possible to perform the method of the invention by hand on the measured and displayed or output signals. For this purpose it is convenient to connect the display unit with a recording device. This may be e.g. a strip chart recorder, which records one or more of the following signals: impedance signal Z (or _{Δ}Z), first time-derivative dZ/dt, EKG-signal. However, the full advantage of the method according to the invention may be utilized if it is automated, which is actually done with the help of the device according to the invention. Furthermore, the relevant data can hardly or not at all be determined with the required accuracy, if done by hand.

The results of the calculation according to the method according to the invention may be displayed on a display 13b. This may come in the form of an alphanumerical display, or a graphic display, in which e.g. the height of a column indicates the calculated values. Furthermore monitors 13a and 13b may be integrated into a single monitor.

The processing unit may also comprise data entering means 15. Like in most computers, the data entering means may be a keyboard, a disk drive, a network connection, a modem, or the like. The data entering means may be used to enter data concerning the patient and/or the measurements. As non-limiting examples, the following data may be entered: the blood resistivity, and patient related data such as name, age, sex, etc.

The method and further parts of the device according to the invention will be explained in connection with FIG. 2 through 5.

In FIG. 2 there is displayed an exemplary measured signal indicative of the impedance signal Z. More precisely, FIG. 2 represents the difference _{Δ}Z between the actually measured value of Z and a mean value Z_{avg}, after which the value of _{Δ}Z has been inverted. In practice, the measured signal Z depends for the greater part on the impedance of the respiratory system (mostly the lung tissue), onto which the more rapidly changing impedance of the heart is superposed. Thus, to improve the accuracy of hand performed measurements and to be better able to perform a visual check on the signals, only _{Δ}Z is shown. But this is not necessary, and it is possible to show the full signal Z.

FIG. 3 shows the first time-derivative dZ/dt of the signal _{Δ}Z. Here, since _{Δ}Z had already been inverted, dZ/dt is also inverted with respect to the actual value. This is done out of custom.

FIG. 4 shows a phonocardiogram, taken with the help of a sound recorder, such as a microphone, and an amplifier. Indicated are the two main sounds, viz. the first heartsound HS1 and the second heartsound HS2. The means for taking a phonocardiogram are not indicated in FIG. 1 for clarity. However, any standard phonocardiogram measuring means may be used.

FIG. 5 shows a typical electrocardiogram (EKG), determined with the help of the EKG-electrodes 9a and 9b and the EKG-measuring means 10. Characteristic peaks in the electrical activity of the heart are visible.

With the help of the signals depicted in the FIG. 2-5 various quantities may be determined, as follows from the method of the invention. The most important characteristics will now be elucidated.

Firstly, in the dZ/dt signal many characteristic features may be indicated. These features basically coincide with the corresponding features in the Z-signal diagram, but there they are much less visible.

The period of a heartbeat does not have a definite beginning or end, but for the purpose of this document a heartbeat is said to start at the beginning of the so-called systolic interval, which marks the contracting phase of the heartbeat. The beginning of systolic interval, or systole, is indicated by both the beginning of the first heart sound HS1 in FIG. 4 and the minimum point 101 in FIG. 3. 104 marks the point where systole ends and, at the same time, the diastolic interval or diastole, begins. The diastolic interval comprises two distinct phases.

The first phase is the isovolumetric relexation time, which runs from point 104 until point 105. This is the phase in which all heartvalves are closed, and no blood enters or leaves the heart. In this period two small peaks are visible in the dZ/dt signal, viz. peaks 110 and 112. It was found by the inventor that these two peaks 110 and 112 relate to the filling of the left, the right coronary artery system, respectively. Experiments showed that by blocking one of the coronary artery systems, the corresponding peaks disappeared completely. On the basis of this knowledge the device and method have been devised.

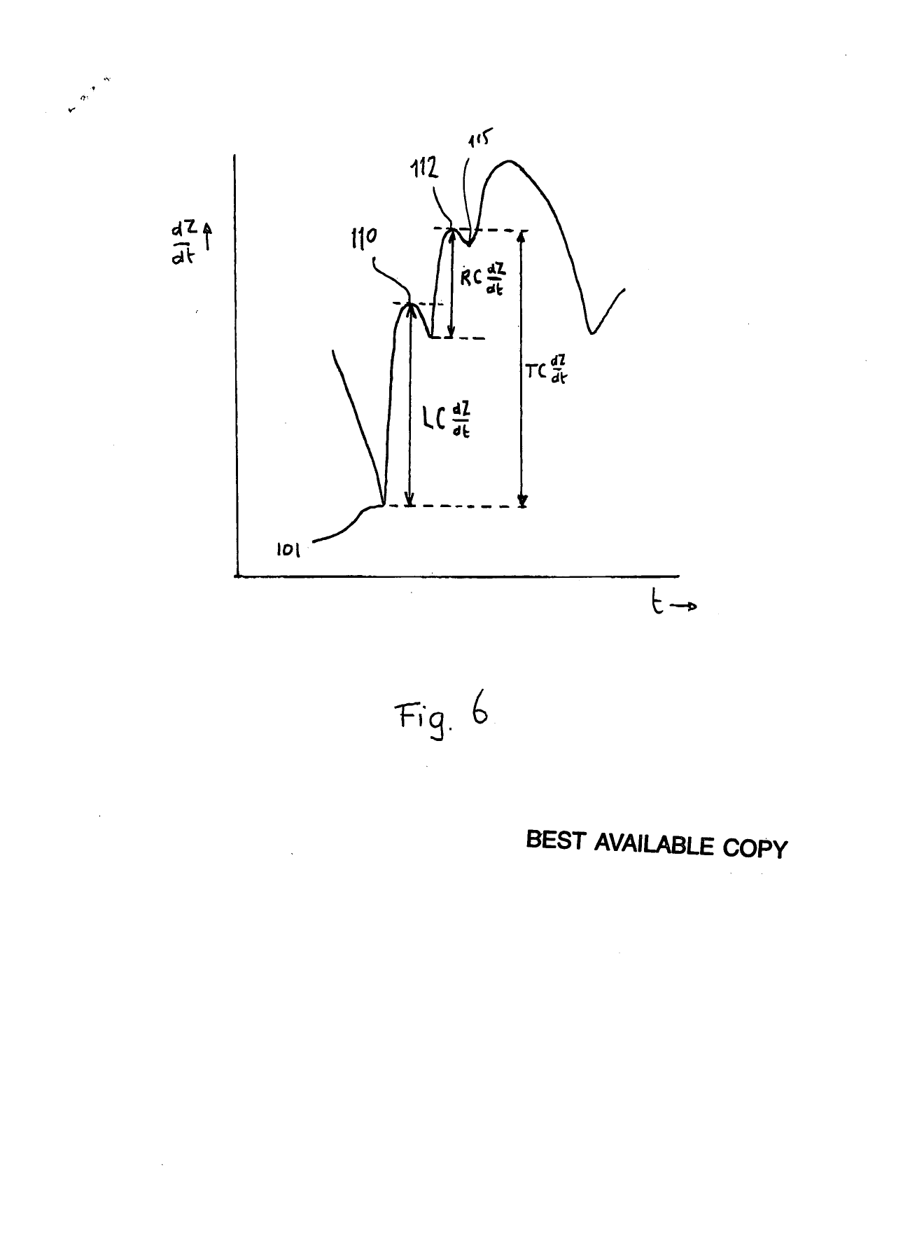

FIG. 6 shows an enlargement of the detail in the box of FIG. 3. Here the peaks 110 and 112 are clearly visible. They are followed by the so-called “O”-wave which begins at point 115.

Peak signal 110 represents the filling of the left half of the coronary artery system, whereas peak 112 represents the filling with blood of the right half of the coronary artery system. The peak height of peak signal 110, i.e. PS1, is indicated with the arrow marked LCdZ/dp, while the peak height of peak 112, i.e. PS2, is marked with the arrow RCdZ/dt. The arrow marked TCdZ/dt indicates a rough estimate of the total blood flow through the total coronary artery system. However it is to be noted that, because of the overlap between the peak signals LCdZ/dt and RCdZ/dt, said signal TC does not represent the actual sum of the left coronary artery blood flow and the right coronary artery blood flow.

It is to be noted that the right coronary artery blood flow signal is only about an ½ to ⅓ times as large as the left coronary artery blood flow signal. This is because of a similar difference in muscle tissue mass and vascular resistance.

Although it is possible to do the calculations according to the method of the invention on peak signals as measured according to FIG. 3, it is much preferred to separate the peak signals PS1 and PS2, i.e. the box as indicated in FIG. 3. This is possible by using a gating circuit. This gating circuit allows to start measuring at a desired point in time. For example, in the measurement it is preferable to start measuring at point 104, which is a minimum value for the first time-derivative dZ/dt. If the measurement is started at point 104, a certain time slot is created, which would run for example from point 104 until the next minimum value for dZ/dt, which is 101′ in the case of FIG. 3. This way, the signal may be represented on a larger scale, because the large maximum value at point 103 is left out. The further separation of peaks 110 and 112 may be done in various ways. For example it is possible to determine the peak height 112 as the value at point 112 minus the minimum value between points 110 and 112. More generally, it is possible that no true minimum is assumed between points 110 and 112. In that case, the value of peak RCdZ/dt may be determined as the value at point 112 minus the value at a point between points 110 and 112, in which dZ/dt suddenly starts to rise again. All these separation techniques may be carried out by hand, or by a computer program, i.e. electronically and thus automatically.

It would also be possible to separate the left and right coronary artery blood flow signals substantially completely. This substantially complete separation might be obtained by establishing separate current fields for the left and right coronary artery system, together with applying measuring electrodes separated in space. This way it is possible to obtain substantially completely separated measuring systems for the left, the right coronary artery system. Because of the relatively high damping factors of the surround (muscle) tissue there is little to no interference when said coronary blood flow signals are measured locally, viz. superficially.

Since in this case only one peak signal PS is measured, the terms “first” and “second” should be interpreted with respect to a complete signal, i.e. one that is measured across all of the heart and with a complete current field.

### EXAMPLE

In a patient study, reference measurements were carried out on a normal patient in a supine position. These included the hematocrit value Hct, the myocardial distance L, the total coronary flow time TCFT, the thoracic impedance Z₀H, the heart rate HR, and, although not shown here, the impedance signal Z as a function of time. From this last quantity the values for the two peak signals PS1 and PS2 have been determined.

From these measured quantities, various other quantities have been calculated according to the invention, such as the constant C, the left and right blood flow volume per beat, and the total coronary flow. Furthermore, with the help of other methods, the total cardiac output, which equals stroke volume times heart rate, has been measured in order to be able to compare the cardiac output and the coronary flows. All these results are shown in the left part of Table 1. For example, it turns out that the left coronary blood flow is larger than the right coronary blood flow by a factor of about 2.3, while the total coronary flow is about 4.3% of cardiac output.

In the right part of Table 1 corresponding measurements are shown, but now for a person during exercise. Many quantities are about the same value, while the blood flow related quantities show a marked increase, they have more than tripled. Still, however, the ratio of left and right coronary flow is about 2.6, while total coronary flow is now 4.9% of cardiac output. Both ratios agree reasonably well with the values during rest. Furthermore, when compared with clinical standards, these values differ by at most 2%, which falls well within the limits of precision of these standards.

TABLE 1

| | Condition 1 (supine, | Condition 2 (during |
|---|---|---|
| | resting) | exercise) |
| Quantity | Value | Value |
| Hct | 0.45 | 0.43 |
| Blood resistivity [Ωcm] | 53.7 | 53.7 |
| Constant C [Ωcm³] | 7733 | 7733 |
| Myocardial distance L [cm] | 12 | 12 |
| TCFT [s] | 0.72 | 0.73 |
| Thoracic impedance Z₀H [Ω] | 14 | 13.7 |
| Value PS1 [Ω/s] | 0.10 | 0.16 |
| Value PS2 [Ω/s] | 0.044 | 0.06 |
| Blood flow QLC [ml/beat] | 2.8 | 4.7 |
| Blood flow QRC [ml/beat] | 1.2 | 1.7 |
| Heart rate HR [beats/min] | 70 | 168 |
| QLC per minute [ml/min] | 198 | 789 |
| QRC per minute [ml/min] | 86 | 296 |
| Total coronary flow [ml/min] | 284 | 1085 |
| Cardiac output [l/min] | 6.6 | 22 |

As mentioned in the discussion of the background of the invention it is repeated here that devices are known which comprise a constant current source, supply electrodes, measuring electrodes, impedance measuring means and some kind of processing unit, in short a bioimpedance measuring device, which device is able to determine the impedance signals necessary to carryout the method of the invention. The device and method according to the invention, however, extend the application of the bioimpedance method to the noninvasive, continuous beat-to-beat monitoring of coronary blood flow signals. Therefore, it could be contemplated that existing bioimpedance measuring devices receive an update in the form of an adapted computer program, an additional computer program or an extension unit which is capable of carrying out the method according to the invention. These existing systems should be accurate enough to be able to determine the peak heights of peaks 110 and 112 or to be able to separate said signals.

Various other modifications of the disclosed embodiments of the invention will become apparent to persons skilled in the art upon reference to the description. It is therefore contemplated that the appended claims will cover such modifications or embodiments as fall within the true scope of the invention.

1. Device for determining at least the blood flow through at least a selected half of a coronary artery system of a beating heart of a mammal, in particular a human being, during a beat of said heart, comprising a bioimpedance measuring device for measuring an impedance signal Z that depends on said blood flow through at least said selected half of said coronary artery system, which bioimpedance measuring device comprises a current source, supply electrodes, one or more upper measuring electrodes, one or more lower measuring electrodes and measuring means, and further comprising processing means which are connected to said measuring means, for processing of at least a value of said impedance signal Z, and for determining a first time-derivative dZ/dt of said impedance signal Z, wherein said processing means further comprise means for separating from said first time-derivative dZ/dt a peak signal PS corresponding to said selected half of said coronary artery system, which peak signal PS lies within a time interval between the beginning of diastole of said heartbeat and the end of a second peak signal PS2 of said first time-derivative dZ/dt which occurs second after said beginning of diastole,
and wherein said processing means are designed to determine said blood flow from said peak signal PS.

2. Device according to claim 1, wherein said processing means are designed to determine said blood flow through said selected half of said coronary artery system from a first peak signal PS1 which occurs first after said beginning of diastole of said heart beat, in the case that said selected half of said coronary artery system is the left half of said coronary artery system of said heart.

3. Device according to claim 1, wherein said processing means are designed to determine said blood flow through said selected half of said coronary artery system from said second peak signal PS2 which occurs second after said beginning of diastole of said heartbeat, in the case that said selected half of said coronary artery system is the right half of said coronary artery system of said heart.

4. Device according to claim 1, wherein the processing means are further designed to determine the duration TCAFT that corresponds to the total coronary artery flow time of said coronary artery system, and further to determine the blood flow volume through said selected half of said coronary artery system during said heart beat, according to
CQ=C/(Z₀H)²·TCAFT·(MCdZ/dt), in which
C=a predetermined constant,
MCdZ/dt=maximum value of said separated peak signal PS, and
Z₀H=value of the impedance of the thorax of said mammal at the time of MCdZ/dt, during said heartbeat,
wherein TCAFT is determined as the time between the beginning of the first peak signal PS1 and the end of diastole of said heartbeat, and
wherein the measuring electrodes have been placed at the midneck region and at the xiphoid junction of said mammal.

5. Device according to claim 4, wherein C has been set equal to C=ρ·(Lm)², in which ρ=the specific resistivity of the blood of said mammal, and Lm=the myocardial distance from an aortic valve of said heart to the apex of said heart.

6. Device according to claim 5, wherein said resistivity ρ is set equal to ρ=53.2·e^{0.022·Hct}, wherein Hct is the hematocrit value of a sample of the blood of said mammal.

7. Device according to claim 4, wherein the processing means are further designed to determine a heart rate in beats per unit time, and further designed to determine a blood flow volume CAQ per unit time through said selected half of said coronary artery system, according to
CAQ=CQ·HR.

8. Device according to claim 4, wherein the processing means are further designed to determine the total blood flow volume through said coronary artery system as the sum of said coronary blood flow volume for said left half of said coronary artery system and said coronary blood flow volume for said right half of said coronary artery system.

9. Method for determining at least the blood flow through at least a selected half of a coronary artery system of a beating heart of a mammal, in particular a human being, during a beat of said heart, comprising the steps of
applying upper and lower measuring electrodes to the body of said mammal, wherein said selected half of said coronary artery system is completely between said upper and lower measuring electrodes,
measuring an impedance signal Z which depends on the blood flow through said selected half of said coronary artery system, by means of a bioimpedance measuring device which is connected to said upper and lower measuring electrodes,
determining a first time-derivative dZ/dt of said impedance signal Z,
separating from said first time-derivative dZ/dt a peak signal PS which corresponds to said selected half of said coronary artery system, which peak signal PS lies within a time interval between the beginning of diastole of said heart during said heartbeat and the end of a second peak signal PS2 of said first time-derivative dZ/dt which occurs second after said beginning of diastole, and
determining said blood flow from said separated peak signal PS.

10. Method according to claim 9, wherein said blood flow through said selected half of said coronary artery system is determined from a first peak signal PS1 which occurs first after said beginning of diastole of said heart beat, in the case that said selected half of said coronary artery system is the left half of said coronary artery system of said heart.

11. Method according to claim 9, wherein said blood flow through said selected half of said coronary artery system is determined from said second peak signal PS2 which occurs second after said beginning of diastole of said heart beat, in the case that said selected half of said coronary artery system is the right half of said coronary artery system of said heart.

12. Method according to claim 9, wherein a blood flow volume CQ during said heartbeat through said selected half of said coronary artery system during said heart beat is determined, according to
CQ=C/(Z₀H)²TCAFT·(MCdZ/dt), in which
C=a predetermined constant,
MCdZ/dt=maximum value of said separated peak signal PS,
Z₀H=value of the impedance of the thorax of said mammal at the time of MCdZ/dt, during said heartbeat, and
TCAFT=total coronary artery flow time=time between the beginning of the first peak signal PS1, and the end of diastole of said heartbeat,
wherein said upper measuring electrodes, lower measuring electrodes, respectively, have been applied at the middle neck region, at the xiphoid junction of the sternum of said mammal, respectively.

13. Method according to claim 12, wherein C is set equal to
C=ρ·Lm², in which
ρ=specific resistivity of the blood of said mammal, and
Lm=the myocardial distance from an aortic valve of said heart to the apex of said heart.

14. Method according to claim 13, wherein said resistivity ρ is set equal to ρ=53.2·e^{0.022·Hct}, wherein Hct is the hematocrit value of a sample of the blood of said mammal.

15. Method according to claim 12, further comprising the steps of determining the heart rate HR in beats per unit time, and determining the blood flow volume CAQ per unit time through said selected half of said coronary artery system, according to
CAQ=CQ·HR.

16. Method according to claim 12, further comprising the step of determining the total value of the blood flow volume through said coronary artery system during said heartbeat as the sum of the blood flow volume through the left half of said coronary artery system and the blood flow volume through the right half of said coronary artery system.

17. Method according to claim 15, further comprising the step of determining the total value of the blood flow volume per unit time through said coronary artery system during said heartbeat as the sum of the blood flow volume per unit time through the left half of said coronary artery system and the blood flow volume per unit time through the right half of said coronary artery system.
